(19) United States
(12) Patent Application Publication
(10) Pub. No.: US 2023/0092974 A1
(43) Pub. Date: Mar. 23, 2023

(54) PROJECTION OF 3D IMAGE DATA ONTO A FLAT SURFACE

(71) Applicant: Siemens Healthcare GmbH, Erlangen (DE)

(72) Inventor: Marcus WAGNER, Fuerth (DE)

(73) Assignee: Siemens Healthcare GmbH, Erlangen (DE)

(21) Appl. No.: 17/948,346

(22) Filed: Sep. 20, 2022

(30) Foreign Application Priority Data
Sep. 21, 2021 (DE) 10 2021 210 504.1

(51) Int. Cl. G06T 19/00; G06T 15/08; G06T 3/00; A61B 6/03

(52) U.S. Cl. CPC G06T 19/00; G06T 15/08; G06T 3/0062; A61B 6/032; G06T 2210/41; G06T 2219/021; G06T 2219/008; G06T 2200/04

(57) ABSTRACT

In a method for transformation of a 3D representation of a part of a body, a central axis of the part of the body to be represented is defined, and a transformation surface that is axis-symmetrical to the central axis is defined. The transformation surface is transformed into a plane and a transformation of the voxels representing the part of the body oriented to the transformed transformation surface is carried out.

### CROSS-REFERENCE TO RELATED APPLICATION(S)

The present application claims priority under 35 U.S.C. § 119 to German Patent Application No. 10 2021 210 504.1, filed Sep. 21, 2021, the entire contents of which are incorporated herein by reference.

### FIELD

One or more example embodiments of the present invention relate to a method for transformation of a 3D representation of a part of the body. Furthermore, one or more example embodiments of the present invention relate to a transformation facility. In addition, one or more example embodiments of the present invention relate to a medical imaging system.

### BACKGROUND

Two- or three-dimensional image data, which can be used for visualization of a mapped examination object and for further applications above and beyond this, is frequently created with the aid of modern imaging methods.

The representation of medical imaging data is mostly carried out in the form of so-called slice images, which overall form a 3D image of an examination region, for example an organ. A problem with the slice images described lies in the fact that that the slices do not allow an overview of the entire examination region or the entire organ, since many structures in the slice image appear obliquely or are arranged at an angle to the slice plane. The user making the examination therefore easily loses the overview or they lose sight of a relationship, which is not so obviously clear from the individual slice images.

Previously 3-dimensional elongated structures, such as for example ribs, vessels or the colon have been visualized by determining a central line referred to as the centerline and by subsequent straightening out together with the surrounding structure. Over and above this unfolding methods exist for the representation of calvaria and the pelvis. On the other hand solid organs which approximately have the shape of an ellipsoid can only be projected onto a plane with difficulty without losing the 3D information.

Described in WO 2011/017 730 is a representation of a skull with 2D data. In this patent the skull is virtually unfolded and projected into one plane.

Described in US 2015/0 131 1881 A1 is a visualization method for a rib cage. In this case the rib cage is virtually unfolded along the spinal cord.

Described in WO 2005/101 323 is a method for creating a panorama image. The method is used in the presentation of hollow organs, such as for example the intestines as part of a colonoscopy.

### SUMMARY

The inventors have identified a problem with regard to clearly representing 3D image data, in particular in the medical field, without losing important information in doing so.

This object is achieved by a method for transformation of a 3D representation of a part of the body, by a transformation facility and by a medical imaging system, according to one or more example embodiments of the present invention.

In the inventive method for transformation of a 3D representation of a part of the body a central axis of the part of the body to be represented is defined. Such a central axis can for example be placed through the geometrical center of mass of the part of the body and extend in the longitudinal direction of the part of the body. By way of illustration it can be said that the object is pierced through with a thin straight line. Moreover a transformation surface axis-symmetrical to the central axis is defined. A part of the body should be understood as a subregion of a body, preferably a geometrically and/or functionally coherent subregion of a body of a patient, for example a human being or an animal. Typical parts of the body comprise organs, bones or similar.

Furthermore a spatial assignment of the voxels representing the part of the body to the axis-symmetrical transformation surface is undertaken, wherein the assignment is undertaken in the radial direction outwards from the central axis. A voxel is to be understood as the three-dimensional equivalent of a pixel. By way of illustration, such a voxel can be expressed as a cuboid cell within a three-dimensional region. The assignment of the voxels in the radial direction can be illustrated as a sampling of the voxels or voxel values, for example gray values or HU values, by a type of beam in the radial direction relative to the central axis, wherein the sampled values are shifted outwards after the sampling onto the transformation surface. Viewed from the central axis outwards, the shifted voxels are subsequently located on the outer side of the transformation surface.

Finally an unfolding transformation is applied to the transformation surface and to the voxels assigned to the transformation surface. In this case the transformation surface is unfolded in one plane and the voxels maintain their spatial assignment to the now unfolded transformation surface during this unfolding. Expressed differently, the voxels are unfolded along with the transformation surface. The voxels form a type of topography on the transformation surface, which is unfolded with the transformation surface. Advantageously the inventive representation allows an overall representation of larger subregions of a part of the body or of the entire part of the body. For example the location and the extent of regions affected by lesions or other defects can be determined more easily. An example of an application of the described inventive method is the representation of a heart perfusion examination. In such a heart perfusion examination a contrast medium is pumped through the coronary vessels. Regions with a lower blood flow, which can indicate constrictions and an infarction, remain dark. In the inventive representation the extent and the location of such a dark region can be easily determined, in particular since the person carrying out the examination can be provided with an overall representation of the heart.

The inventive transformation facility has an axis-positioning unit for defining a central axis of a part of the body to be represented. A surface-positioning unit for defining a transformation surface axis-symmetrical to the central axis is also part of the inventive transformation facility.

Furthermore the inventive transformation facility comprises an assignment unit for spatial assignment of the voxels representing the part of the body to the axis-symmetrical transformation surface in the radial direction outwards from the central axis.

The inventive transformation facility also comprises a transformation unit for application of an unfolding transformation to the transformation surface and to the voxels assigned to the transformation surface, wherein the transformation surface is unfolded in one plane and the voxels maintain their spatial assignment to the now unfolded transformation surface. The inventive transformation facility shares the advantages of the inventive method.

The inventive medical imaging system, preferably a computed tomography system or an MR system, has a scan unit for acquisition of raw data or measurement data from an examination object. The medical imaging system can moreover have a control facility that, as well as a plurality of control functions, can also comprise the function of a reconstruction unit in order to carry out an evaluation of the acquired measurement data and to reconstruct image data on the basis thereof. Also part of the inventive medical imaging system is an inventive transformation facility. The inventive transformation facility can in particular be part of the said control facility of the medical imaging system. The inventive medical imaging system shares the advantages of the inventive transformation facility.

The major components of the inventive transformation facility can predominantly be embodied in the form of software components. This relates in particular to the axis-positioning unit, the surface-positioning unit, the assignment unit and the transformation unit. In principle however these components can also be realized in part, in particular when it is a matter of fast computations, in the form of software-supported hardware, for example FPGAs or the like. Likewise the interfaces needed, for example if it is only a matter of transferring data from other software components, can be embodied as software interfaces. They can however also be embodied as interfaces constructed from hardware, which are activated by suitable software.

A largely software-based realization has the advantage that even computer units and/or control facilities of medical imaging systems already used beforehand can be upgraded in a simple manner by a software update in order to work in the inventive way. To this extent the object is also achieved by a corresponding computer program product with a computer program, which is able to be loaded directly into a memory facility of a computer unit or a control facility of a medical imaging system and comprises program sections for carrying out all steps of the inventive method when the computer program is executed in the computer unit or control facility of the medical imaging system.

Such a medical imaging system, as well as the computer program, can where necessary comprise additional elements such as e.g. documentation and/or additional components, as well as hardware components, such as e.g. hardware keys (dongles etc.) for use of the software.

For transport to the memory facility of a computer unit of a medical imaging system and/or for storage on the computer unit of the medical imaging system, a computer-readable medium, for example a memory stick, a hard disk or another transportable or permanently installed data medium can be used, on which the programmable and executable program sections of the computer program able to be read in and executed by a computer unit are stored. For this purpose the computer unit can for example have one or more interoperating microprocessors or the like for example.

The dependent claims and also the description given below each contain especially advantageous embodiments and developments of the present invention. In this case the claims of one claim category can in particular also be developed in a similar way to the dependent claims of another claim category. Moreover, within the framework of the present invention, the various features of different exemplary embodiments and claims can also be combined to form new exemplary embodiments.

Preferably, in the inventive method for transformation of a 3D representation of a part of the body, the transformation surface comprises a projection surface. Furthermore the transformation of the voxels comprises a projection of the voxels of the 3D representation of the part of the body onto the projection surface. In this variant the voxels are initially projected outwards onto a projection surface. Voxels placed after one another in a radial direction are projected in this case onto projection surfaces lying coaxially in relation to one another at a distance of one voxel. The projection surfaces in this case preferably lie outside the object to be imaged. Advantageously the three-dimensional object is transformed into a multiplicity of slices lying conformant to the projection surfaces, which are subsequently transformed into flat slices and in this way show a type of topography or relief map of a three-dimensional object.

Especially preferably in the inventive method voxels arranged in one projection direction of the 3D representation of the part of the body are shifted along the projection direction by the distance between the central axis and the projection surface. Through the shift the three-dimensional object, put simply, is spread out or stretched over the projection surface, so that in a subsequent step it can be “pressed flat” in order to obtain a type of relief or slice representation.

It is quite especially preferred for the shifting of the voxels of the object to occur in such a way that the voxels are subsequently arranged positioned with regard to the central axis on the far side of the projection surface. As already mentioned the part of the body to be represented is spread out so far that the projection surface virtually represents the base surface in the case of a conversion of the projection surface into one plane and a corresponding transformation of the voxels.

In this case, after the shifting of the voxels, there is a transformation of the projection surface into one plane and the voxels are arranged with the same transformation assigned to the plane. Advantageously the voxels now form a type of relief of the object to be represented, which in particular is useful for a clear representation of hollow organs, since their entire wall surface and wall depth can be clearly shown.

Likewise in the inventive method the transformation preferably comprises a reformation. In this variant the transformation surface comprises a multiplicity of cut surfaces. The cut surfaces in this case are arranged coaxially around the central axis. The cut surfaces define slices lying between two neighboring cut surfaces, which are cut in the axial direction and transformed into a flat slice in each case. Especially preferably the slices are of the same thickness, i.e. the distances between the individual cut surfaces are constant. Advantageously a slice stack with equal slice thickness is created. The values of the individual voxels can be sampled in a similar way to a ray cast method radially outwards from the central axis. The position of the individual voxels in one slice plane can then be determined as a function of the distance of the respective voxel from the central axis and also as a function of a position of the penetration point of the ray on which the respective voxel is located through the cut surfaces.

In this variant the projection or shifting of the voxels outwards or the stretching of the part of the body to be represented on a projection surface is omitted. Instead slices arranged coaxially about the central axis are created, which in a second step are transformed into flat slices and can be displayed as a type of slice stack.

It is quite especially preferred in the inventive method for the transformation surface to comprise a cylinder outer surface. In this case annular slices arranged coaxially about the central axis are produced that, put simply, can be cut and “unrolled” and finally arranged stacked on one another and together form a slice stack. The individual slices have a different extent depending on their distance from the central axis. To put it simply, the slice assigned directly to the central axis corresponding to the small circumference of the cylinder outer surface of the transformation surface is the smallest while the outermost slice corresponding to the circumference of the cylinder outer surface of the transformation surface has the greatest extent.

The inventive method is preferably applied to the pictorial representation of a part of the body that comprises a hollow organ. Advantageously the entire outer layer of the hollow organ can be represented as a map by the inventive transformation, so that the extent and position of individual regions becomes easily recognizable.

The hollow organ shown preferably comprises one of the following organs:
the heart,
the bladder,
the prostate,
the uterus,
the lungs,
the stomach,
the thyroid.

As already mentioned, individual subregions of the hollow organs can be localized more exactly and their extent easily determined. Examples of this are the localization and delimitation of regions with weak blood flow, tumors or other pathological phenomena in the said hollow organ.

### BRIEF DESCRIPTION OF THE DRAWINGS

The present invention will be explained again below in greater detail by referring to the enclosed figures with the aid of exemplary embodiments. In the figures:

FIG. 1 shows a schematic diagram of a view from above of an organ, which is created by a multiplicity of projections within the framework of the method for transformation of a 3D representation of a part of the body in accordance with an exemplary embodiment of the present invention,

FIG. 2 shows the organ already shown in FIG. 1 in a representation projected onto one plane,

FIG. 3 shows a view from above of the organ shown in FIG. 1 both in the original representation and also in a representation projected onto a cylinder surface,

FIG. 4 shows a view from above of the organ projected onto the cylinder surface during the unfolding of the cylinder surface into one plane,

FIG. 5 shows a perspective view of a multiplicity of two-dimensional slices of the organ shown in FIG. 1 to FIG. 4,

FIG. 6 shows a diagram of a method for transformation of a 3D representation of a part of the body in accordance with an alternate exemplary embodiment of the present invention,

FIG. 7 shows a flow diagram, which illustrates a method for transformation of a 3D representation of a part of the body in accordance with an alternate exemplary embodiment of the present invention,

FIG. 8 shows a schematic diagram of a transformation facility in accordance with an exemplary embodiment of the present invention,

FIG. 9 shows a schematic diagram of a medical imaging system in accordance with an exemplary embodiment of the present invention.

### DETAILED DESCRIPTION

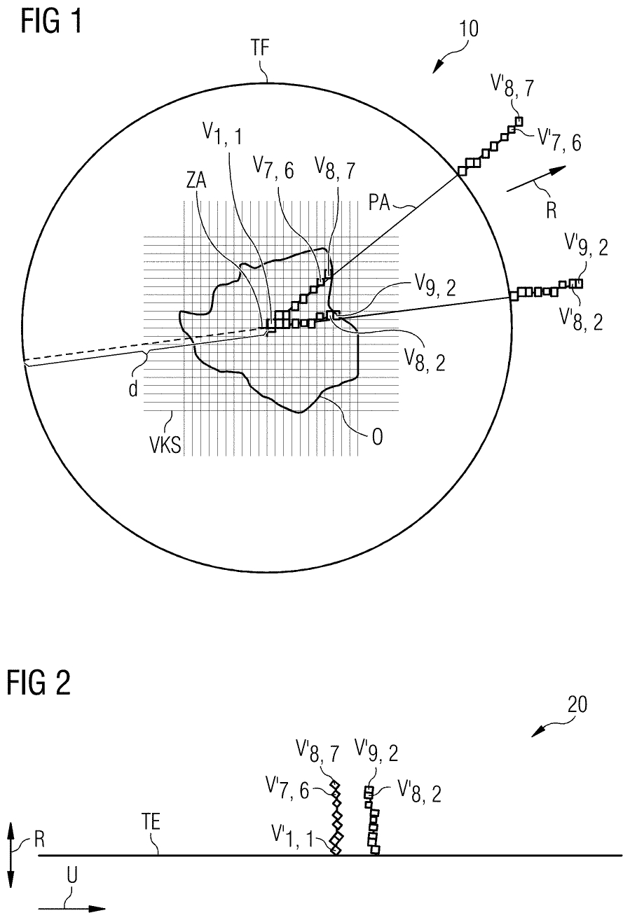

Shown in FIG. 1 is a schematic cross-sectional view 10 from above onto an organ O. The organ shown in FIG. 1 is intended to represent a heart, which is shown in a view from above. The organ is shown in a grid VKS (VKS=voxel coordinate system) consisting of voxels V. Placed through the center of the organ O is a vertical central axis ZA, which is oriented at right angles to the plane of the page, and a projection cylinder TF is placed about the central axis ZA at a distance d. The voxels V_{1,1} lying directly around the central axis ZA are now projected onto this projection cylinder or cylinder outer surface ZA in radial direction R. Voxels V_{7,6}, V_{8,2}, V_{8,7}, V_{9,2} lying further outwards are projected onto a projection cylinder lying beyond the first projection cylinder TF, lying coaxial to the central axis ZA (not shown), so that “towers” of voxels V_{7,6}′, V_{8,2}′, V_{8,7}′, V_{9,2}′ growing in the projection direction, i.e. in the radial direction, are produced on the first projection cylinder TF.

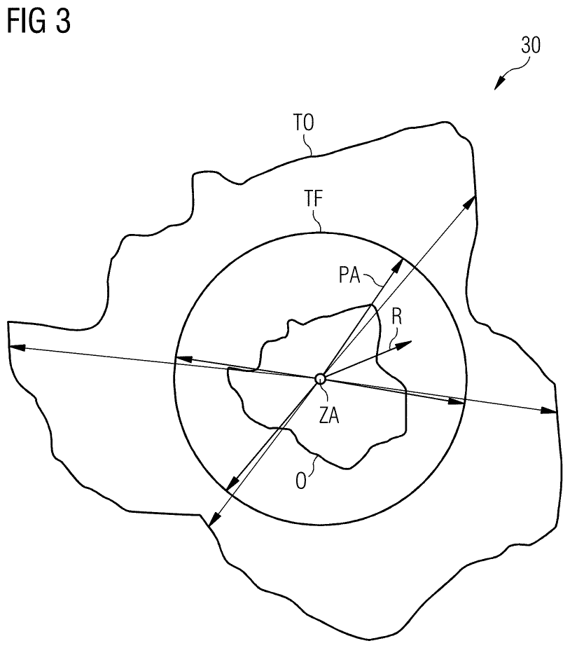

Shown in FIG. 2 is a schematic diagram 20, wherein the projection cylinder TF is unfolded in a circumferential direction U to a plane TF and the voxels V_{1,1}′, V_{7,6}′, V_{8,2}′, V_{8,7}′, V_{9,2}′ already shown in FIG. 1 can be seen here as voxel “towers”. The fact that the transformation surface, i.e. the projection cylinder TF, has now been transformed into a plane means that the voxel “towers” now stand in parallel to one another and form a type of relief. This cannot be seen directly in FIG. 2, since for the sake of clarity only two of the voxel “towers” are shown in this diagram.

Shown in FIG. 3 is a view 30 of an organ O from above corresponding to that shown in FIG. 1, which is now projected entirely onto the projection cylinder TF or onto the further projection cylinder (not shown) lying at a voxel distance from it. Thus while the representation shown in FIG. 1 and FIG. 2 shows only a few examples of projected voxels, all voxels of the organ O projected onto the projection cylinder TF or onto the further projection cylinder lying about said cylinder are shown in FIG. 3. To put it simply, the organ O has been mapped by an expansion to a transformed organ TO. Thus a plurality of projection axes PA are indicated in FIG. 3, with which voxels are projected, so that the transformed organ TO, to put it simply, is stretched out on the projection cylinder TF mapped in FIG. 3.

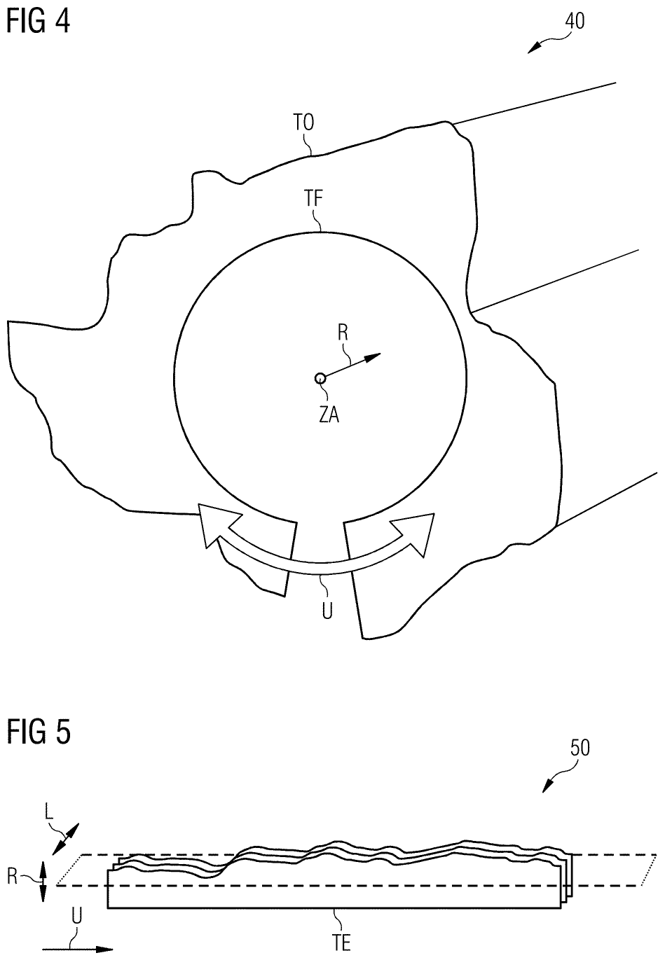

Illustrated in FIG. 4 is a snapshot 40 of how the organ already projected in FIG. 3 is now unfolded. By way of illustration in this case the inner projection cylinder TF is transformed into a plane TE, wherein the projection cylinder TF is cut up in the longitudinal direction and unfolded in the circumferential direction U.

The result can be seen in a diagram 50 in FIG. 5. Shown there in a perspective representation is a plane TE of length L and of width U, wherein the length L corresponds to the length of the central axis ZA through the organ O and the width U to the circumference of the projection cylinder TF. The topography of the organ O is shown in the radial direction R in FIG. 5.

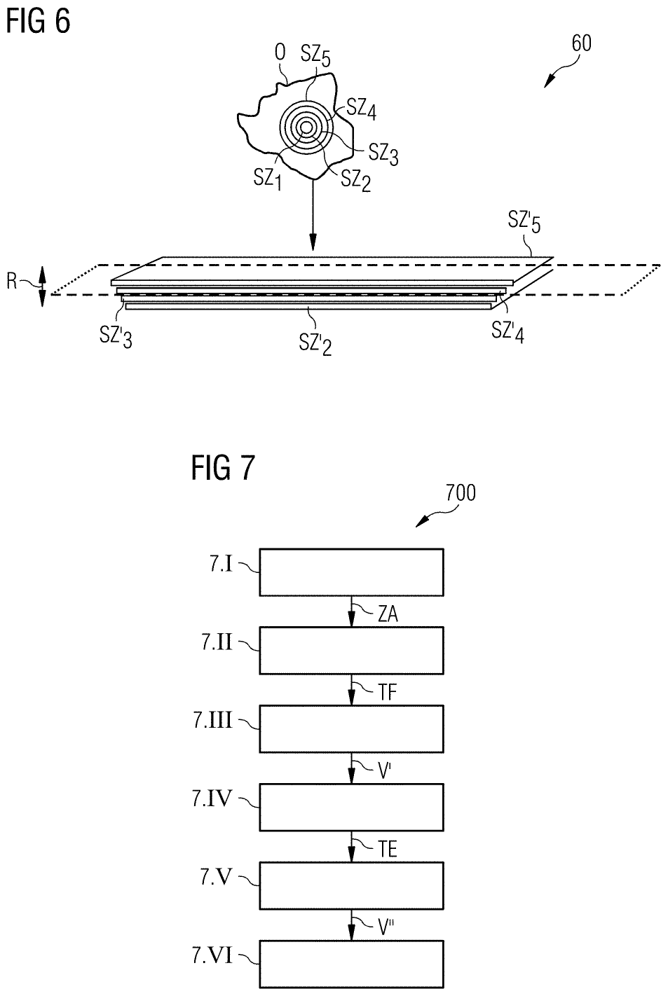

Shown in FIG. 6 is a schematic diagram 60 of a procedure for an alternate method in accordance with invention. In this variant there is initially no projection onto cylinder surfaces, but instead the cylinder surfaces are brought into the organ O itself running coaxially about the central axis ZA form cut surfaces SZ₁, SZ₂, SZ₃, SZ₄, SZ₅, at which the organ O is virtually divided up into cylinder outer surface-shaped slices. The cylinder outer surfaces SZ₁, SZ₂, SZ₃, SZ₄, SZ₅ are subsequently unfolded to flat surfaces, which delimit layers with a different extent. These layers form the structure of the organ O in the radial direction R virtually as a profile in the vertical direction.

Shown in FIG. 7 is a flow diagram 700, which illustrates a method for transformation of 3D representation of a part of the body in accordance with an exemplary embodiment of the present invention. In the step 7.I, first of all, a central axis ZA is placed through the organ to be mapped. The location of such a central axis ZA is selected for example so that the central axis ZA runs through the geometrical center of mass of the organ. Furthermore the central axis ZA can be oriented in the vertical direction.

In the step 7.II a projection cylinder is embodied as a transformation surface TF about the central axis ZA, wherein the central axis ZA at the same time forms the cylinder axis of the projection cylinder.

In the step 7.III mappings V′ of the voxels V of the organ O are created, wherein the mapped voxels V′ are arranged on the transformation surface TF in the manner shown in FIG. 1.

In the step 7.IV the transformation surface TF is transformed into a plane TE and in the step 7.V the mapped voxels V′ arranged on the transformation surface TE are mapped as voxels V″ onto the plane TE and there embody a type of profile of the object O in the radial direction relative to the central axis ZA.

Finally, in the step 7.VI, there is a display of the created profile of the object O.

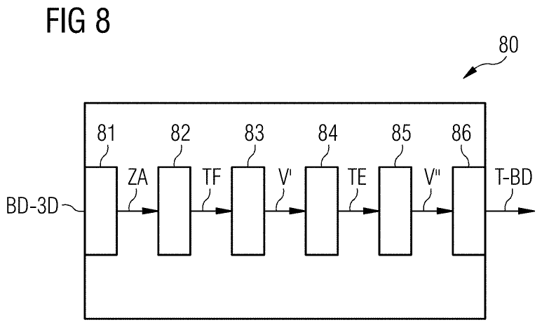

Illustrated in FIG. 8 is a schematic diagram of a transformation facility 80 in accordance with an exemplary embodiment of the present invention.

The transformation facility 80 shown in FIG. 8 comprises an axis-positioning unit 81, which is configured to receive three-dimensional image data BD-3D of an organ, which originates from a reconstruction unit for example, and to define a central axis ZA through the mapped organ. A surface-positioning unit 82, which defines a transformation surface TF, which is arranged axis-symmetrically to the central axis ZA, is also part of the transformation facility 80. The transformation facility 80 also comprises an assignment unit 83. The assignment unit 83 is configured to assign the voxels V representing the organ O to the axis-symmetrical transformation surface TF, wherein the assignment is made in the radial direction outwards from the central axis ZA. In this case transformed voxels V′ are created. A surface transformation unit 84, which is likewise part of the transformation facility 80, is configured to create a transformed, i.e. a flat, transformation surface TE. On the basis of this transformation a voxel transformation unit 85 creates twice-transformed voxels V″, which represent a profile of the organ in the radial direction on the flat transformation surface TE. Finally the transformation facility 80 also comprises a display unit 86 for output of transformed slice image data T-BD, as are shown for example in FIG. 5.

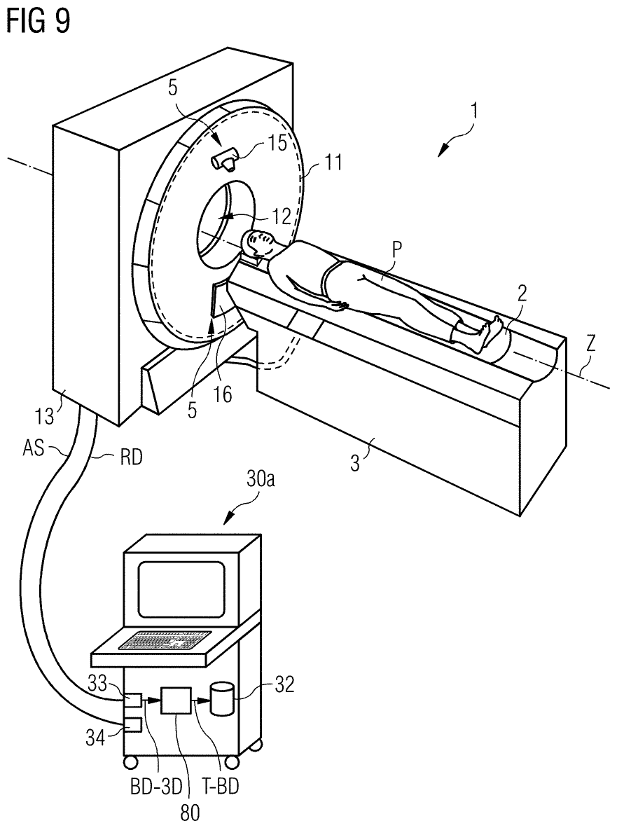

Shown in FIG. 9 is a computed tomography system 1, known for short as a CT system 1, which comprises the transformation facility 80 shown in FIG. 8. The CT system 1 essentially consists in this case of the usual scan unit 13, in which on a gantry 11, a projection measurement data acquisition unit 5 with an x-ray detector 16 and an x-ray source 15 lying opposite the detector 16 orbit around a measurement space 12. Located in front of the scan unit 13 is a patient support facility 3 or a patient table 3, of which the upper part 2 with a patient P located thereon can be pushed into the scan unit 13 in order to move the patient P through the measurement space 12 relative to the detector system 16. The scan unit 13 and the patient table 3 are controlled by a control facility 30a, from which acquisition control signals AS come via a usual control interface 34 in order to control the system as a whole in accordance with predetermined measurement protocols in the conventional manner. In the case of a spiral acquisition a helical path is produced by a movement of the patient P in the z direction, which corresponds to the system axis z longitudinally through the measuring space 12 and to the simultaneous circulation of the x-ray source 15 relative to the patient P during the measurement. The detector 16 always moves in parallel opposite the x-ray source 15 in this case, in order to acquire projection measurement data RD, which is then used for reconstruction of volume and/or slice image data BD-3D. Likewise a sequential measurement method can also be carried out in which a fixed position in the z-direction is moved to and then, during an orbit, a part orbit or a number of orbits at the z position concerned, the required projection measurement data RD is acquired, in order to reconstruct a slice image at this z position or in order to reconstruct image data BD-3D from the projection measurement data of a number of z positions. The inventive method is in principle also able to be used on other CT systems, e.g. with only a single x-ray source and with an opposing x-ray counting detector or with a single x-ray source with a kV switching function or an x-ray detector forming a complete ring. For example the inventive method is also able to be applied to a system with a stationary patient table and gantry movable in the z direction (known as a sliding gantry).

The projection measurement data RD acquired from the detector 16 (also called raw data below) is transferred to a reconstruction unit 33 of the control facility 30a. The reconstruction unit is realized in this exemplary embodiment in the control facility 30 in the form of software on a processor. Subsequently the created three-dimensional image data BD-3D is transformed by the transformation facility 80 in the way described in conjunction with FIG. 1 to FIG. 8.

The transformed image data T-BD created by the transformation facility 80 is then stored in a memory 32 of the control facility 30a and/or output in the usual way on the screen of the control facility 30a. It can also be read via an interface not shown in FIG. 9 into a network connected to the computed tomography system 1, for example a radiological information system (RIS), and held in mass storage accessible there or output as images on printers or filming stations connected there. In this way the data can be further be processed in any given way and then stored or output. Via the control facility 30a there is also a determination of suitable activation parameters or activation signals AS on the basis of previously entered data, in particular of measurement parameter values A and information about the type of imaging. The activation signals are subsequently transferred to the said control interface 34. From there the activation signals AS are then transferred directly to the units involved in the imaging, such as for example the x-ray source 15, the x-ray detector 16, the patient support 3 etc.

The components of the transformation facility 80 can be realized predominantly or completely in the form of software elements on a suitable processor. In particular the interfaces between the components of the transformation facility 80 or between the transformation facility 80 and other components of the control facility 30a can also be embodied purely as software. The only requirement is that access options to suitable memory areas exist, in which the data can be suitably buffered and can be retrieved and updated again at any time.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

In conclusion it is pointed out once again that the aforementioned method and apparatuses merely involve preferred exemplary embodiments of the present invention and that the present invention can be varied by the person skilled in the art, without departing from the area of the present invention, provided it is predetermined by the claims. Thus the method for transformation of a 3D representation of a part of the body and the transformation facility 80 has primarily been explained with the aid of a system for recording medical image data. The present invention is however not restricted to an application in the medical area, but the present invention in principle can also be applied for the recording of images for other purposes. For the sake of completeness it is also pointed out that the use of the indefinite article “a” or “an” does not exclude the features involved also being able to be present a number of times. Likewise the use of the term “unit” does not exclude said unit consisting of a number of components, which if necessary can also be spatially distributed.

What is claimed is:

1. A method for transformation of a 3D representation of a part of a body, the method comprising:
defining a central axis of the part of the body;
defining a transformation surface that is axis-symmetrical to the central axis;
spatially assigning voxels representing the part of the body to the transformation surface, wherein the spatially assigning is undertaken in a radial direction outwards from the central axis;
applying an unfolding transformation to the transformation surface and the voxels assigned to the transformation surface, wherein
the transformation surface is unfolded in a plane and the voxels maintain their spatial assignment to the unfolded transformation surface.

2. The method as claimed in claim 1, wherein
the transformation surface includes a projection surface, and
transformation of the voxels includes projecting the voxels representing the part of the body onto the projection surface.

3. The method as claimed in claim 2, wherein the voxels representing the part of the body that are arranged in a projection direction are shifted in the projection direction by a distance between the central axis and the projection surface.

4. The method as claimed in claim 3, wherein the voxels representing the part of the body that are arranged in a projection direction are shifted such that the voxels are subsequently arranged in relation to the central axis positioned outside of the projection surface.

5. The method as claimed in claim 3, wherein, after shifting of the voxels, the method comprises:
transforming the projection surface into a plane, and
assigning the voxels to the plane with a same transformation.

6. The method as claimed in claim 1, wherein
the unfolding transformation includes a reformation,
the transformation surface includes a multiplicity of cut surfaces,
the cut surfaces are arranged coaxially about the central axis, and
the cut surfaces define slices lying between neighboring cut surfaces, which are cut in an axial direction and transformed into a flat slice.

7. The method as claimed in claim 1, wherein the transformation surface includes a cylinder outer surface.

8. The method as claimed in claim 1, wherein the part of the body includes a hollow organ.

9. The method as claimed in claim 8, wherein the hollow organ comprises one of
a heart,
a bladder,
a prostate,
a uterus,
lungs,
a stomach, or
a thyroid.

10. A transformation facility, comprising:
an axis-positioning unit configured to define a central axis of a part of a body to be represented;
a surface-positioning unit configured to define a transformation surface that is axis-symmetrical to the central axis;
an assignment unit configured to spatially assign voxels representing the part of the body to the transformation surface, wherein the voxels representing the part of the body are assigned to the transformation surface in a radial direction outwards from the central axis; and
a transformation unit configured to apply an unfolding transformation to the transformation surface and to the voxels assigned to the transformation surface, wherein
the transformation surface is unfolded in a plane and the voxels maintain their spatial assignment to the unfolded transformation surface.

11. A medical imaging system, comprising:
a scanner configured to acquire raw data from an examination object;
a reconstruction unit configured to reconstruct image data based on the raw data; and
a transformation unit as claimed in claim 10.

12. A non-transitory computer program product including a computer program, which is loadable into a memory of a computer device, the computer program including program sections that, when executed by the computer device, cause the computer device to carry out the method of claim 1.

13. A non-transitory computer-readable medium storing program sections that, when executed by at least one processor, cause the at least one processor to perform the method of claim 1.

14. The method as claimed in claim 4, wherein, after shifting of the voxels, the method comprises:
transforming the projection surface into a plane, and
assigning the voxels to the plane with a same transformation.

15. The method as claimed in claim 2, wherein
the unfolding transformation includes a reformation,
the transformation surface includes a multiplicity of cut surfaces,
the cut surfaces are arranged coaxially about the central axis, and
the cut surfaces define slices lying between neighboring cut surfaces, which are cut in an axial direction and transformed into a flat slice.

16. The method as claimed in claim 3, wherein
the unfolding transformation includes a reformation,
the transformation surface includes a multiplicity of cut surfaces,
the cut surfaces are arranged coaxially about the central axis, and
the cut surfaces define slices lying between neighboring cut surfaces, which are cut in an axial direction and transformed into a flat slice.

17. The method as claimed in claim 4, wherein
the unfolding transformation includes a reformation,
the transformation surface includes a multiplicity of cut surfaces,
the cut surfaces are arranged coaxially about the central axis, and
the cut surfaces define slices lying between neighboring cut surfaces, which are cut in an axial direction and transformed into a flat slice.

18. The method as claimed in claim 5, wherein
the unfolding transformation includes a reformation,
the transformation surface includes a multiplicity of cut surfaces,
the cut surfaces are arranged coaxially about the central axis, and
the cut surfaces define slices lying between neighboring cut surfaces, which are cut in an axial direction and transformed into a flat slice.

19. The method as claimed in claim 6, wherein the transformation surface includes a cylinder outer surface.

20. An apparatus for transforming a 3D representation of a part of a body, the apparatus comprising:
a memory storing computer readable instructions; and
at least one processor configured to execute the computer readable instructions to cause the apparatus to
define a central axis of a part of a body to be represented,
define a transformation surface that is axis-symmetrical to the central axis,
spatially assign voxels representing the part of the body to the transformation surface, wherein the voxels representing the part of the body are assigned to the transformation surface in a radial direction outwards from the central axis, and
apply an unfolding transformation to the transformation surface and to the voxels assigned to the transformation surface, wherein
the transformation surface is unfolded in a plane and the voxels maintain their spatial assignment to the unfolded transformation surface.
